(19) United States
(12) Patent Application Publication
(10) Pub. No.: US 2012/0035340 A1
(43) Pub. Date: Feb. 9, 2012

(54) AMINATED HEMICELLULOSE MOLECULE AND METHOD FOR PRODUCTION THEREOF

(75) Inventors: Rikard Slättegård, Vällingby (SE); Marcus Ruda, Bandhagen (SE); Harry Brumer, Johanneshov (SE)

(73) Assignee: SWETREE TECHNOLOGIES AB, Umeå (SE)

(21) Appl. No.: 13/144,914

(22) PCT Filed: Feb. 5, 2010
(86) PCT No.: PCT/SE2010/050138

§ 371 Date: Sep. 29, 2011

Related U.S. Application Data
(60) Provisional application No. 61/150,021, filed on Feb. 5, 2009.

(30) Foreign Application Priority Data
Feb. 5, 2009 (SE) 0950058-8

(51) Int. Cl. C08B 37/14; C07H 5/06; C07H 1/06

(52) U.S. Cl. 527/312; 536/55.3; 536/18.7

(57) ABSTRACT

The present invention relates to a method for reductive amination of a water soluble carbohydrate. An aminated water soluble carbohydrate is a prerequisite in processes for further modification of cellulose. The synthesis of this molecule comprises, providing a water soluble carbohydrate, an amine and a reducing agent, which are reacted under acidic conditions, isolated to give an aminated water soluble carbohydrate with a yield larger than 60%. The invention also relates to an aminated hemicellulose molecule with a molecular weight of at least 1 kDa, especially xyloglucan.

### TECHNICAL FIELD

The present invention relates to a method for reductive amination of a water soluble carbohydrate. The synthesis of this molecule comprises, providing a water soluble carbohydrate, an amine and reducing agent, which are reacted under acidic conditions to give an aminated water soluble carbohydrate with a yield larger than 60%. The invention also relates to an aminated hemicellulose molecule with a molecular weight of at least 1.0 kDa, especially xyloglucan.

### BACKGROUND OF THE INVENTION

Reductive amination is a chemical reaction which involves the conversion of a carbonyl group to an amine via an intermediate imine. The carbonyl group is most commonly a ketone or an aldehyde. Reductive amination is also possible for carbohydrates with a is reducing end (hemiacetal), see scheme 1.

The reductive amination of carbohydrates with a reducing end can be performed in one pot, with the imine formation and reduction occurring concurrently. This is known as direct reductive amination, and is carried out with a reducing agent that is stable in water and reactive in acidic conditions, e.g. sodium cyanoborohydride (NaBH₃CN), see scheme 1.

Methods for reductive amination of carbohydrates are needed in many applications. One example is to use reductive amination to modify xyloglucan and xyloglucan oligosaccharides, which can be further used for modification of cellulose or cellulosic materials.

In the patent literature such as the invention by Charmot et al. U.S. Pat. No. 7,030,187, methods for modification of cellulosic material with polymers are described. This patent discusses a reductive amination route where the terminal glucose residue on a cellulosic material is converted with an amine, with either sodium borohydride or with sodium cyanoborohydride, and reduction under high pressure of hydrogen according to four references, Danielsson et al., Larm et al., WO98/15566 and EP 0725082.

Inventors at Univ. of Georgia have shown that a dye chemically attached to xyloglucan can be used for dyeing of fabrics, US2006/0242770. In this process they attach the amino dye to the reducing end of xyloglucan oligosaccharides (XGO) via reductive amination.

In all described processes to get a modified cellulose material there is a need to get a xyloglucan (XG) or xyloglucan oligosaccharides (XGO) modified at the reducing end by reductive amination. The process must be very efficient with high yield and low costs. Scheme 2 shows the reductive amination on xyloglucan fragments.

Currently Presented Methods for Reductive Amination of Carbohydrates.

A commonly used standard method for introducing molecules into carbohydrates is by reductive amination, whereby the reducing end of the carbohydrate is converted by an excess of a primary amine into an imine, which is then reduced to obtain an amine.

To obtain a free primary amine at the reducing end in one step (1-amino-1-deoxy sugars), the carbohydrate is usually treated with ammonium salts and a reducing agent e.g sodium cyanoborohydride. Scheme 3, shows reductive aminations on xyloglucan fragments using ammonium carbonate and sodium cyanoborohydride.

These procedures for reductive amination of xyloglucan oligosaccharide are low yielding (2-50%) and the reaction times are long about 6-20 days Danielsson and Grey 1986, Fry et al. Plant J. 1997; Bourquin et al. Plant cell., 2002, and Brumer et al. J. Am. Chem. Soc. 2004.

The purification procedures are also time consuming and expensive and are not compatible for large scale synthesis. The reductive amination of XGO are usually carried out in water with large excess of ammonium salts and a reducing agent, 100-150 equivalents ammonium hydrogencarbonate and 10-20 equivalents of sodium cyanoborohydride Brumer et al. J. Am. Chem. Soc. 2004. These salts have to be removed from the reaction mixtures by ion exchange chromatography, size exclusion chromatography or by dialysis, which all are costly when used in large industrial settings.

Some authors indicate that the pH is important in the initial reaction when using primary organic amines. In US2006/0242770 the reaction is made acidic with acetic acid to obtain a pH of 3 to 4, in one of the experiments in this US patent application a pH of 3.85 is used. According to Evangelista et al. 1996 the pH should be below 3, which is reached by adding acetic acid. Yoshida and Lee 1994 use glacial acetic acid to reach the pH of 6-9 with a pH optimum 7 in their reaction settings. Furthermore Schwartz & Gray 1977 have show that the pH optimum is in the range of 8-9. This part of the reaction is performed at an elevated temperature.

Each of these steps is subject to acid catalysis, and the improvement in rate in the presence of acid [Evangelista et al, 1996] is not in itself surprising. The degree of acidity required for effective reductive amination of sugars is, however, much greater than that required for simple aldehydes, suggesting that the acid catalysis is required. The reactivity of the reducing agents e.g. sodium cyanoborohydride is also increased when acidic conditions are used.

It should be noted that in the literature many of the reductive amination reactions are done on monosaccharides, such as mannose, glucose, NAc-glucose, galactose or disaccharides such as lactose. Only a few examples have been found where larger oligosaccharides are used, such as trisaccharides and tetrasaccharides.

A summary of a few presented methods is found in table 1.

In none of the presented process the yield was sufficient for an industrial scale at an acceptable low costs. Thus, there is a need for a method to produce carbohydrates that are aminated at the reducing end with high reproducibility and high yield. Furthermore it is needed that the reductive amination of the carbohydrate can be done at a low cost.

### SUMMARY OF THE INVENTION

A new procedure for the reductive amination of water soluble carbohydrates has been developed, which is superior from previously presented standard procedures. The synthesis is a one pot procedure, where the carbohydrate is dissolved and then reacted with an amine, such as benzylamine, 4,4′-diaminostilbene-2,2′-disulfonic acid (an optical brightening agent (OBA)), 4-nitroaniline, dodecylamine or allyl amine under reducing conditions. During the process development it was unexpected that the yield became higher than 60% and even as high as 89 to 98%. An aminated hemicellulose molecule, especially xyloglucan with a molecular weight of at least 1 kDa is produced.

The invention is described together with the following figures:

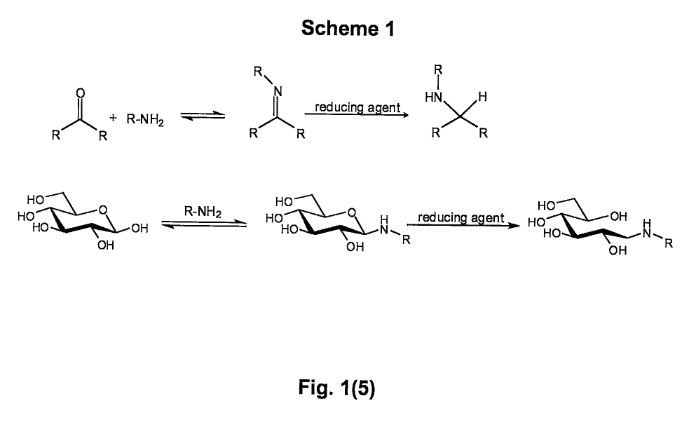

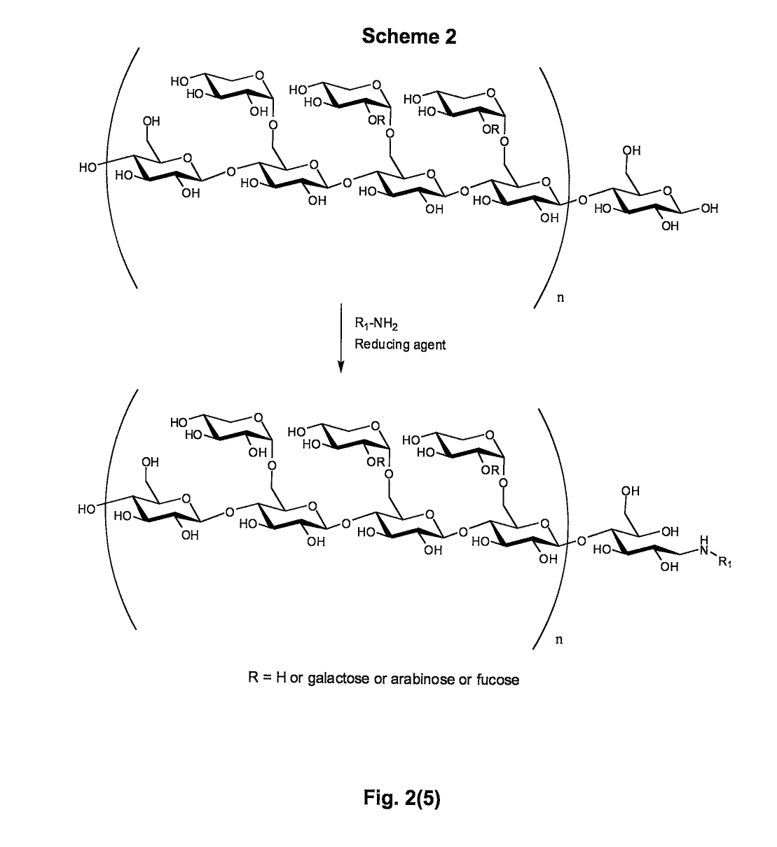

FIG. 1: Reaction scheme 1 prior art

FIG. 2: Reaction scheme 2 according to the invention

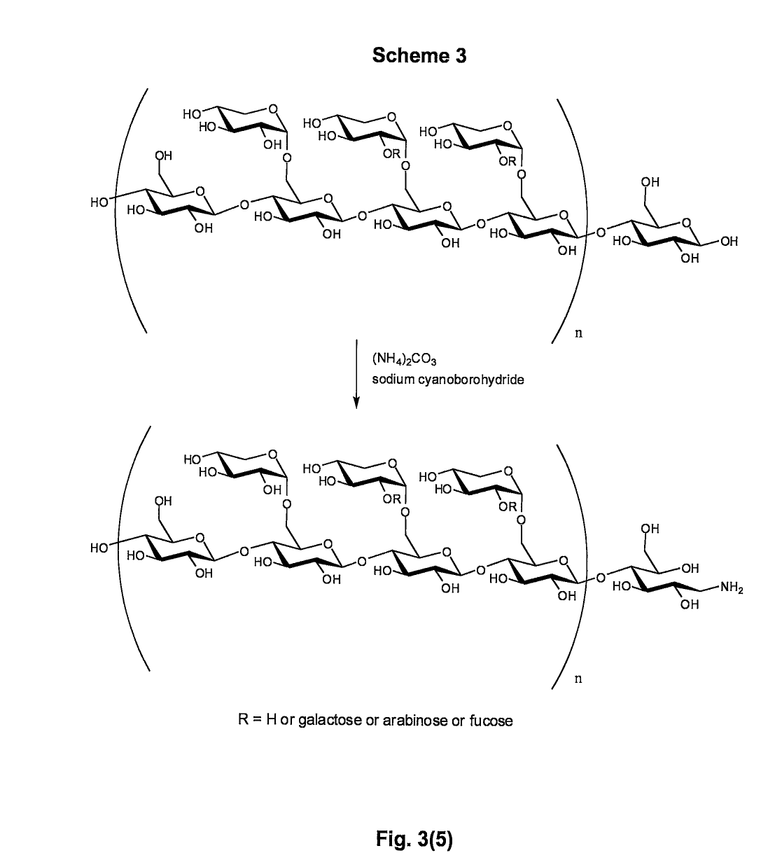

FIG. 3: Reaction scheme 3 prior art

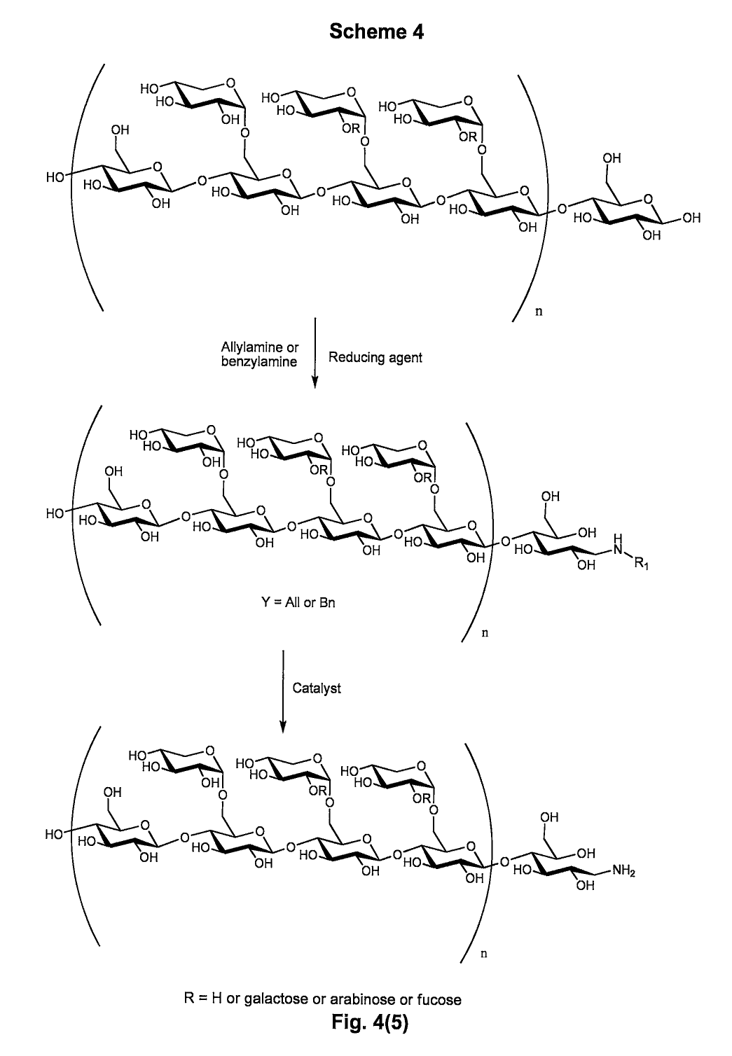

FIG. 4: Reaction scheme 4 according to the invention

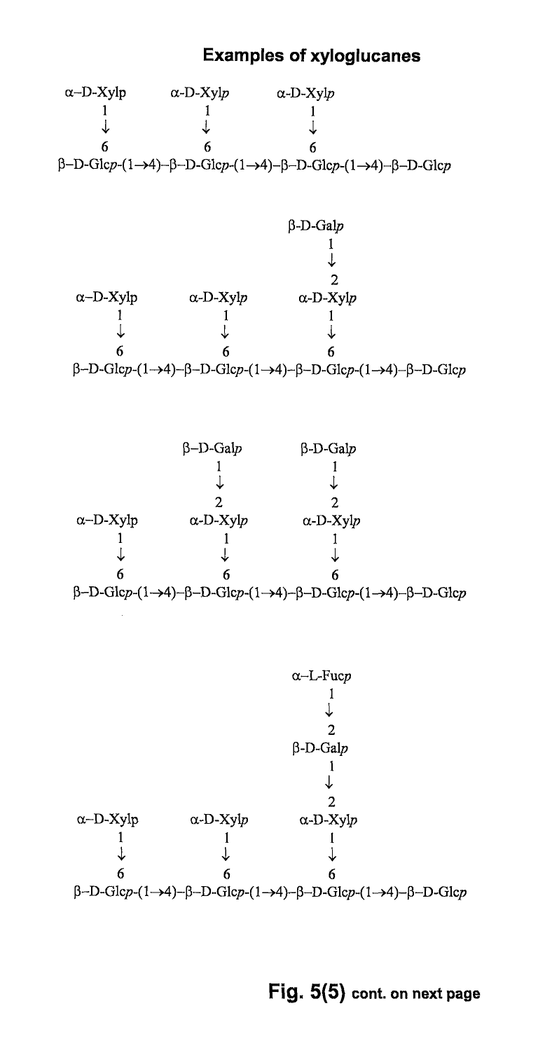

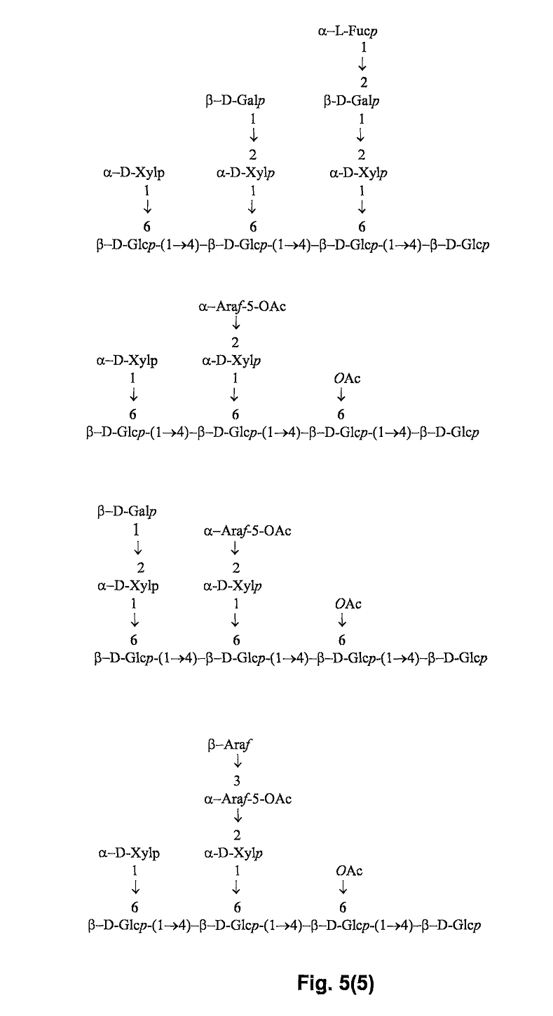

FIG. 5: Examples of xyloglucoses

### DETAILED DESCRIPTION OF THE INVENTION

A method was invented to produce an aminated water soluble carbohydrate in a reaction mixture comprising, a water soluble carbohydrate and an amine under reducing conditions
I) where the reaction mixture is made acidic (adjusted to a pH<7) and then incubated,
II) after the incubation the generated aminated water soluble carbohydrate may be precipitated.

The primary amine in the reaction has the formula R₁NH_{2.} This will produce a secondary amine according to Scheme 2.

R₁ is a chemical group, which may be removable or not removable from the amine.

According to one embodiment the R₁ group is removed and a primary amino group is created on the carbohydrate. This may be done by hydrolysing the aminated carbohydrate or by isomerising and hydrolysing of the aminated carbohydrate.

Thus, the invention also relates to a method further comprising the steps of removing the R₁ group and creating an amino group on the carbohydrate by
III) hydrogenolysing the generated aminated carbohydrate by
   a) dissolving the generated aminated carbohydrate in water and acid and reacting it with hydrogen gas at an elevated pressure in the present of an catalyst,
   b) removing the catalyst
   c) where after the aminated water soluble carbohydrate product may be precipitated
or
IV) isomerisation and hydrolysation of the aminated carbohydrate by
   a) dissolving the generated aminated carbohydrate in water and acid and reacting it in the present of an catalyst,
   b) removing the catalyst
   c) where after the aminated water soluble carbohydrate product may be precipitated

The invention also relates to a method further comprising the steps of removing the R₁ group and creating a primary amino group on the carbohydrate (scheme 4). When R₁ is a benzyl group by
III) hydrogenolysing the generated aminated carbohydrate by
   a) dissolving the generated aminated carbohydrate in water and acid and reacting it with hydrogen gas at an elevated pressure in the presence of an catalyst,
   b) removing the catalyst
   c) where after the aminated water soluble carbohydrate product may be precipitated

A further variant of the invented method when R₁ is an allyl group.
IV isomerisation of the allyl group to an enamine and hydrolysis by
   a) dissolving the generated aminated carbohydrate in water and acid and reacting it in the presence of an catalyst,
   b) removing the catalyst
   c) where after the aminated water soluble carbohydrate product may be precipitated

Process step I in the above cited methods is performed without the addition of salts e.g. without the addition of ammonium salts.

Other amines such as 2,4-Dimethoxybenzylamine, 4-Methoxybenzylamine, or 2,4,6-Trimethoxybenzylamine, which are cleavable under acidic conditions, may be used in stead of benzylamine or allylamine to introduce other removable R₁ groups.

Other amines with cleavable R₁ groups can be used, which a person that is skilled in the art can recognize.

The secondary amine obtained in step I) may be used as it is obtained in the reaction mixture or be precipitated and isolated before reacted further in steps III) or IV) to be transformed into a primary amine.

In a preferred embodiment of the invented method to produce an aminated water soluble carbohydrate, the yield of the generated aminated water soluble carbohydrate is higher than 60%. This relates to step I) as such when a secondary amine is produced and also to the combination of step I) with step III) or IV) converting the secondary amine to a primary amine.

In one embodiment of the invented method the reducing condition in step I) may be a hydrogen atmosphere and a catalyst such as platinum, platinum derivatives or a reducing agent that preferably are stable in water e.g. sodium cyanoborhydride, sodium dithionite or amine borane complexes such as pyridine borane, dimethylamine borane or 2-picoline borane.

The reducing agent and the carbohydrate may be used in an equivalent ratio of 100 to 1, 90 to 1, 80 to 1, 70 to 1, 60 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1.5 to 1, 1.2 to 1, 1.1 to 1, 1 to 1 preferably 1.2 to 1.

In a preferred embodiment of the invented method to produce an aminated water soluble carbohydrate the amine in step I) is a primary organic amine. Primary amines according to the invention have the formula R₁H₂N, wherein R₁ represents alkyl and aromatic groups. The alkyl groups may be chosen from alkyl groups with 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 carbon atoms. The alkyl groups may be strait, branched or cyclic they may be saturated or unsaturated such as alkenes and alkynes e.g. with the above stated number of carbon atoms. The alkyl group may be a cyclic saturated group with 6 ring atoms that may comprise one or more heteroatoms such as O. One or more such cyclic saturated groups with 6 ring atoms that may comprise one or more heteroatoms such as O may be bound together. They may be fused together or bound to each other by glycosidic bonds.

The aromatic groups may be chosen from those with between 3 and 14 ring atoms. They may be mono-, bi- and polycyclic and comprise carbon atoms only as ring atoms or one or more hetero atoms chosen from N, S and O.

Example of hetero aromatic groups are Furan, Benzofuran, Isobenzofuran, Pyrrole, Indole, Isoindole, Thiophene, Benzothiophene, Benzo[c]thiophene, Imidazole, Benzimidazole, Purine, Pyrazole, Indazole, Oxazole, Benzoxazole, Isoxazole, Benzisoxazole, Thiazole, Benzothiazole. They may be six membered such as Benzene, Pyridine, Pyrazine, Pyrimidine and Pyridazine. The may be fused bicycic rings such as Naphthalene, Quinoline, Quinoxaline, Isoquinoline, Quinazoline and Cinnoline and fused polycyclic rings such as Anthracene, Acridine and Acridine.

The alkyl and aryl groups may be substituted with one or more groups chosen from OH, NH₂, Cl, I, Br, F, alkyl with the number of carbon atoms as defined above, alkoxy with the number of carbon atoms as defined above, e.g. methoxy.

Further R₁ may be, carbonyl containing derivatives, phosphorus derivatives, silicon containing compounds, boron containing compounds, selenium containing derivatives, sulphur containing derivatives, alcohol containing derivatives, ether containing derivatives, epoxide containing derivatives, heterocycles, acetal containing compounds, —NH-alkyl derivatives, —NH-Aryl derivatives, —NH-Benzyl derivatives, —NH—CO-alkyl derivatives, —NH—CO-aryl derivatives, —NH—CO-Benzyl derivatives. For example 4,4′-diaminostilbene-2,2′-disulfonic acid (OBA), benzylamine, allylamine, dodecyl amine or 4-nitro aniline.

In a preferred embodiment of the invented method to produce an aminated water soluble carbohydrate the water soluble carbohydrate is a hemicellulose, especially xyloglucan.

In a preferred embodiment of the invented method to produce an aminated water soluble carbohydrate the water soluble carbohydrate is xyloglucan oligosaccharides.

In another embodiment of the invented method to produce an aminated water soluble carbohydrate the carbohydrate comprises at least three monosaccharides, such as trisaccharides.

In another embodiment of the invented method to produce an aminated water soluble carbohydrate the water soluble carbohydrate is a soluble carbohydrate, which comprises at least 4, at least 5, at least 6, at least 7, at least 8, at least 9 or at least 10 monosaccharides, e.g. from 4 to 10500, from 4 to 13500 monosaccharides.

In another embodiment of the invented method to produce an aminated water soluble carbohydrate the water soluble carbohydrate is a xyloglucan with a molecular weight of at least 1 kDa, at least 1.35 kDa, at least 1.4 kDa, at least 2 kDa, at least 3 kDa, at least 4 kDa, or at least 10 kDa, such as from 1 kDa to 1.5 million, e.g. from 1 kDa to 1.5 million, from 1.35 kDa to 1.5 million, from 1.4 kDa to 1.5 million, from 2 kDa to 1.5 million, from 3 kDa to 1.5 million, from 4 kDa to 1.5 million, from 10 kDa to 1.5 million.

In another embodiment of the invented method to produce an aminated water soluble carbohydrate the water soluble carbohydrate is a xyloglucan with a molecular weight of 1 kDa to 1.5 million Da, e.g. of 1350 Da up to 50 kDa.

It is generally difficult to measure the molecular weight of polysaccharides. The above figures represent the average molecular weight.

In some embodiments of the invention starch, dextrane, dextrin, agarose or sepharose is not included as water soluble carbohydrates.

In one embodiment of the invented method to produce an aminated water soluble carbohydrate the pH in reaction mixture in I) is adjusted to about pH 5.

In another embodiment of the invented method the pH in the reaction mixture in step I) should have a pH of from pH 4.0 to pH 6.0 such as pH 4.0, 4.1, 4.2, 4.3, 4.4, 4.5, 4.6, pH 4.7, pH 4.8, pH 4.9, pH 5.0, pH 5.1, pH 5.2, pH 5.3, pH 5.4, pH 5.5, pH 5.6, pH 5.7, pH 5.8 or pH 5.9.

In another embodiment of the invented method the pH in the reaction mixture in step I) should not be pH 4.5 or less then pH 4.5. In another embodiment of the invented method the pH in the reaction mixture in step I) should not be pH 6 or higher than pH 6.

In another embodiment of the invented method the pH in the reaction mixture in step I) is above pH 4.5 and below pH 6.

In one embodiment of the invention the reaction mixture may be made basic (pH>7) after step I, where after the generated aminated water soluble carbohydrate may be precipitated.

The pH in reaction mixture after step 1) may be adjusted to a pH that is above the pKₐ value of a protonated amine e.g. from pH7 to pH12, such as about pH 7, pH 7.5, pH 8, pH 8.5, pH 9, pH 9.5, pH 10, pH 10.5, pH 11, pH 11.5 or pH 12, e.g. pH9-10, such as pH9. Aqueous ammonia may be used.

The acid used in step I and III) a) and IV) a) may be any inorganic acid, such as HCl or any organic acid, such as acetic acid.

In steps I, II, III and IV the reaction solvent may be water or substantially water dissolving substantially most of the ingredients.

In a yet another embodiment of the invented method to produce an aminated water soluble carbohydrate an alcohol with 1-4 carbon atoms such as methanol, ethanol, 1-, 2-propanol, 1-, 2-, 3-butanol, preferably methanol is added in the reaction mixture in step I) before incubation.

A mixture of an alcohol and water is preferably used as solvent, because this gives a high yield. A ratio of 1-10:1, especially of 4:1 between alcohol and water may be used. According to one embodiment a ratio of 4:1 of methanol and water is used.

After the optional addition of an alcohol preferably methanol to the reaction mixture in step I) and substantial completion of the reaction, the alcohol may be removed, e.g. by evaporation. If pH is made basic, the alcohol may be removed before the pH is adjusted and made basic.

The substantial completion of the reaction in step I), III) and IV) may be decided by e.g. TLC (thin layer chromatography), MALDI-TOF, NMR, IR or GPC.

In a preferred embody the yield is at least 60%, at least 65%, at least 70%, at least 75%, at least 80%, at least 85%, at least 90%, at least 95%, or even close to 100%, such as between 60% and 100% or in any interval created by the combination of any of the above mentioned percentage figures.

In another embodiment of the method steps I) and IV) are performed at an elevated temperature. Accordingly, the temperature may be about room temperature, 25° C., but also 30° C., 35° C., 40° C., 45° C., 50° C., 55° C., 60° C., 65° C., 70° C., 75° C., 80° C., 85° C., 90° C., 95° C. or 100° C. The temperature may lie between room temperature and 100° C. or in any interval created by the combination of any of the above mentioned temperature figures.

In a preferred embodiment of the method the temperature in I) is about 55° C.

In another embodiment of the method the incubation time in I) is over night.

In another embodiment of the method the incubation time in I) lies between 1 and 100 hours such as about 12, 14, 16, 18, 20, 22, 24, 48, 60, 72 or 84 hours.

In another embodiment of the method the incubation time in I) is about 18 hours.

In a preferred embodiment of the invented method a catalyst is added in step IIIa) and IVa).

In a preferred embodiment of the invented method, the catalyst in step IIIa) and IV) may be palladium on activated carbon. The catalyst may comprise 2%-25% palladium on activated carbon.

In a preferred embodiment of the invented method, the added catalyst in step IIIa) and IV) is 10% palladium on activated carbon.

In a preferred embodiment of the invented method, the added catalyst in step IIIb) and IVb) is removed before precipitation of the compound.

In one embodiment of the invented method, the precipitation may be done with an aprotic organic solvent such as acetone or a protic organic solvent such as any alcohol, especially ethanol.

The synthesis procedure may comprise a reaction mixture where the carbohydrate is condensed with an primary amine, such as benzylamine or allylamine under reducing conditions.

Instead of using large amounts of primary amines, 1.5 equivalents of primary amines is preferably used together with 1.5 equivalents of sodium cyanoborohydride. A mixture of an alcohol such as methanol and water is preferably used as solvent, because this gives a high yield. A ratio of 4:1 between these solvents resulted in a good solubility of the starting materials and the highest yields.

The reaction is especially carried out in slightly acidic conditions, which gave better results compared to basic conditions. A pH of 5, which was obtained by the addition of acetic acid gave the best results. The almost salt free reaction conditions simplified the purification and the reagents and by products were removed by extraction and evaporation. The products were isolated by precipitation and the white solids were collected by centrifugation.

Other reducing agents such as triacetoxy borohydride, amine boranes such as pyridine borane, dimethylamine borane or 2-picoline borane, sodium dithionite or platinum oxide together with a hydrogen atmosphere can replace the reducing conditions created by sodium cyanoborohydride.

The synthesis procedure comprises a reaction mixture where the carbohydrate is condensed with an amine, such as benzylamine or allylamine under reducing conditions In a further reaction the benzyl group and allyl group respectively may be cleaved e.g. by hydrolysis and/or catalysis leading to carbohydrates with primary amino groups, see scheme 4. Catalytic amount of palladium on activated carbon is preferably used as a catalyst in the cleavage step, and this catalyst can be reused to save both the environment and to reduce the production cost in this step.

In the hydrogenolysis reaction in step IIIa), the hydrogenolysis may be carried out in acidic conditions to increase the reaction rate. An organic acid (acetic acid) was used, since it can easily be removed from the product during the precipitation and centrifugation steps. To further increase the rate of this reaction higher hydrogen pressure may be used. A hydrogen pressure of 6-10 psi has been used, but a pressure of at least 20, at least 30, at least 40, at least 50, at least 60, at least 70, at least 80, at least 90, or at least 100 psi may be used, even much higher pressure can be used such as 200, 500, 700 or 1000 psi. Thus, a pressure of 6-1000 psi may be used or an interval created by a combination of any of the above mentioned pressure figures, such 6-10 psi.

The invention also relates to a method for introducing an amino group in a water soluble carbohydrate comprising all steps I, III) and IV) wherein the incubation step I), may be performed without limitation as to pH. Thus, this method may be performed at any pH, in the incubation step I) preferably at the above mentioned pH values.

Thus, the invention relates to a method to produce an aminated water soluble carbohydrate in a reaction mixture comprising, a water soluble carbohydrate and an amine with the formula R₁NH₂, wherein R is a chemical group removable from an amine,
I) where the reaction mixture is incubated,
II) then the generated aminated water soluble carbohydrate may be precipitated, removing the R₁ group e.g. a benzyl group from the amine by
III) hydrogenolysing the generated aminated carbohydrate by
   a) dissolving the generated aminated carbohydrate in water and acid and reacting it with hydrogen gas at an elevated pressure in the presence of an catalyst,
   b) removing the catalyst
   c) where after the aminated water soluble carbohydrate product may be precipitated
      or removing the R₁ group e.g an allyl group from the amine by
IV) isomerisation of the aminated carbohydrate to an enamine and removing the enamine by acid hydrolysis.
The isomerisation and hydrolysis of the aminated carbohydrate in step IV) may be performed by
   a) dissolving the generated aminated carbohydrate in water and acid and reacting it in the present of an catalyst
   b) removing the catalyst
   c) where after the aminated water soluble carbohydrate product may be precipitated.

All information and details mentioned in this specification regarding steps II, III and IV apply mutatis mutandis to this procedure as well and vice versa, the only difference being that there may be no restriction as to pH in step I. Thus all step II), III) and IV) may be performed under the same condition irrespective of what pH is used in step I).

Several advantages of this method are identified such as; that small amount of benzylamine (1 to 3, preferably 1.5 equivalents) and NaCNBH₃ (1 to 3, preferably 1.5 equivalents) are used. Catalytic amount of palladium on activated carbon is preferably used as a catalyst in the hydrogenolysis step, and this catalyst can be reused to save both the environment and to reduce the production cost in this step.

Furthermore, the process comprises several parts that are favourable when transferring to large scale settings. Since the reaction contains very little salts, reagents can be removed e.g. by evaporation and by extraction into organic solvents and standard industrial scale equipment can be used for precipitation and centrifugation.

The synthesis can be performed in large scale and in high yields 75-98% compared to previous methods. The reaction time in the first step is about 18 hours at an elevated temperature of 55° C., this reaction time is much shorter compared to the about 3 to 20 days used before. This is especially remarkable since this is applicable on polysaccharides with high molecular weights.

Furthermore it was unexpected that the reaction setting in step I) had an optimal pH 5, since earlier reports claim that the pH should be below pH 4 or about pH 7-8 and that the yield was between 89 and 98%. This became further unexpected when compared to the experiments presented in US 2006/0242770 (WO2004/094646) where they used both pH 4 (pH 3.85 in ex. 1) and pH 4.5 (acetate buffer in ex. 4) and the yield was about 40%.

The invention also relates to an aminated hemicellulose molecule, with a molecular weight of at least 2 kDa, especially a xyloglucan molecule. Especially the invention relates to an aminated hemicellulose molecule produced by the method herein described and comprising the above defined amine groups.

According to one embodiment the molecular weight of the aminated hemicellulose molecule is at least 1 kDa, at least 1.35 kDa, at least 1.4 kDa, at least 2 kDa, at least 3 kDa, at least 4 kDa, or at least 10 kDa, such as from 1 kDa to 15 kDa, e.g. from 1 kDa to 15 kDa, from 1.35 kDa to 15 kDa, from 1.4 kDa to 15 kDa, from 2 kDa to 15 kDa, from 3 kDa to 15 kDa, from 4 kDa to 15 kDa, from 10 kDa to 15 kDa.

The invention also relates to an aminated hemicellulose molecule having the formula:

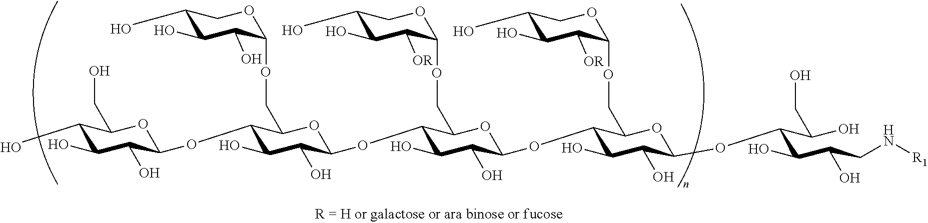

wherein:

R₁ is any chemical group and where R is H, galactose, arabinose or fucose.

n is above 1 such as between 2 and 2000 and the molecular weight is above 1350 Da

n is between 2 and 2000

and the molecular weight is above 6500 Da.

What has been stated above regarding the molecular weight of the aminated hemicellulose produced with the method of the invention also relates to the molecules of the above formula.

All cited publications are incorporated as references.

The invention is supported by the following non limiting examples.

### EXAMPLE 1-5

XGO were used and the reactions with sodium cyanoborohydride as the reducing agent, scheme 2.

### EXAMPLE 1

R₁=benzyl group (Bn)

XGO (50 g, 39 mmol) was dissolved in MeOH/H₂O (4:1, 700 mL) at 55° C. Benzylamine (6.4 mL, 59 mmol) and NaCNBH₃ (3.7 g, 59 mmol) were added. Acetic acid (7 mL) was then added to obtain a pH of 5 and the reaction mixture was stirred at 55° C. over night. After completion of the reaction according to TLC (acetonitrile/H₂O 2:1) and MALDI-TOF the reaction mixture was evaporated to remove methanol. The products were then precipitated in cold ethanol (3 L) and the white solids were collected by centrifugation. The products were dried under vacuum to give 49 g of the products (36 mmol, 92%). (R₁=Bn).

### EXAMPLE 2

R₁=Allyl group (All)

XGO (3 g, 2.34 mmol) was dissolved in a mixture of 25 mL MeOH, 5 mL H₂O and 0.30 mL of acetic acid. NaCNBH₃ (0.220 g, 3.5 mmol) and allylamine (0.277 mL, 3.69 mmol) were added and the reaction was stirred at 55° C. over night. After completion according to TLC (acetonitrile-H₂O 2:1) and MALDI-TOF the mixture was precipitated in ice-cold ethanol. The white material was collected by centrifugation and dissolved in water and concentrated. Freeze drying gave 2.65 mg of the product (0.20 mmol, 85%). (R₁=All).

### EXAMPLE 3

R₁=4,4′-diaminostilbene-2,2′-disulfonic acid (OBA)

XGO (8.18 g, 6.4 mmol) was dissolved in 80 mL water. 4,4′-diaminostilbene-2,2′-disulfonic acid (23.2 g, 62.7 mmol) and NaCNBH₃ (2.36 g, 37.6 mmol) were added and the pH of the mixture was adjusted to 6 with 1M NaOH (15 mL). The mixture was stirred for 48 h and then filtered (glass filter) to remove excess of 4,4′-diaminostilbene-2,2′-disulfonic acid. To the filtrate 1M HCl (25 mL) was added, until pH reached 2-3. The mixture was purified by filtration through a plug of reversed phase silica gel (C-18). Concentration and freeze-drying gave 12.57 g of the product, which contained minor impurities of unreacted 4,4′-diaminostilbene-2,2′-disulfonic acid. (R₁=4,4′-diaminostilbene-2,2′-disulfonic acid).

### EXAMPLE 4

R₁=4-nitroaniline

4-nitroaniline (55 mg, 0.40 mmol) was added to a mixture of XGO (0.1 g, 0.08 mmol) in methanol (3 mL). NaCNBH₃ (25 mg, 0.40 mmol) was added and a few drops of HCl (1M) to obtain a pH of 6. The reaction was stirred at 50° C. for 72 h when MALDI-TOF and TLC (acetonitrile-H2O 2:1) showed full conversion to the products (R₁=4-nitroaniline).

### EXAMPLE 5

R₁=dodecyl

XGO (1.0 g, 0.78 mmol) was dissolved in 10 mL H₂O. NaCNBH₃ (74 mg, 1.17 mmol) and dodecylamine was added (173 mg, 0.94 mmol. Acetic acid was added to obtain a pH of 5 and the reaction was stirred at 55° C. for 48 hours. After completion according to MALDI-TOF the product was precipitated in ice-cold ethanol and the white material was collected by centrifugation. The precipitate was dissolved in water and the ethanol was removed by evaporation. The product was freeze dried to give 0.73 g (0.50 mmol, 64%) of the product (R₁=dodecyl).

### EXAMPLE 6-9

Reductive amination of XGO with benzylamine using different reducing agents, scheme 2.

### EXAMPLE 6

Reducing agent=sodium triacetoxyborohydride, Na(OAc)₃BH

XGO (1 g, 0.78 mmol) was dissolved in 4 mL MeOH and 1 mL H₂O. Na(OAc)₃BH (0.331 g, 1.56 mmol) and benzylamine (0.17 mL, 1.56 mmol) were added. Acetic acid was added to obtain a pH of 5 and the reaction was stirred at 55° C. over night. More Na(OAc)₃BH (0.331 g, 1.56 mmol) and benzylamine (0.17 mL, 1.56 mmol) was added and the mixture was stirred for 48 hours at 55° C. After completion according to TLC (acetonitrile-H₂O 2:1) and MALDI-TOF the mixture was precipitated in ice-cold ethanol. The white material was collected by centrifugation and dissolved in water and concentrated. The product was precipitated again in ethanol and the isolation of the product was repeated. Freeze drying gave 616 mg of the product (0.48 mmol, 62%). (R₁=Bn)

### EXAMPLE 7

Reducing agent=dimethylamine borane complex ((CH₃)₂NH BH₃)

XGO (1 g, 0.781 mmol) was dissolved in a mixture of 8 mL MeOH, 2 mL H₂O and 0.1 mL of acetic acid. Benzylamine (0.128 mL, 1.17 mmol) and the dimethylamine borane complex (0.220 g, 3.5 mmol) were added and the reaction was stirred at 30° C. over night. According to TLC only small amounts of products was formed. The mixture was stirred for 72 more hours at 40° C. with three more additions of dimethylamine borane complex (3×70 mg) to complete the reaction according to TLC (acetonitrile-H₂O 2:1) and MALDI-TOF. The mixture was concentrated and the product was precipitated in ice-cold ethanol. The white material was collected by centrifugation and dissolved in water and concentrated. Freeze drying gave 788 mg of the product (0.58 mmol, 74%). (R₁=Bn).

### EXAMPLE 8

Reducing agent=2-picoline borane complex (C₆H₇N BH₃)

XGO (1 g, 0.781 mmol) was dissolved in a mixture of 8 mL MeOH, 2 mL H₂O and 0.1 mL of acetic acid. Benzylamine (0.128 mL, 1.17 mmol) and picoline borane complex (125 mg) were added and the reaction was stirred at 40° C. over night. According to TLC only small amounts of starting material was left and more picoline borane complex was added (125 mg) and the mixture was stirred over night again at 40° C. TLC (acetonitrile-H₂O 2:1) and MALDI-TOF indicated now complete conversion and the reaction was stopped. The mixture was concentrated and the product was precipitated in ice-cold ethanol. The white material was collected by centrifugation and dissolved in water and concentrated. Freeze drying gave 747 mg of the product (0.55 mmol, 70%). (R₁=Bn).

### EXAMPLE 9

Reducing agent=sodium dithionite

0.5 g XGO was dissolved in 2 ml of 0.5 M NaOAc buffer (pH=5.5) followed by addition of 84 mg (2 eq.) benzyl amine and 0.27 g (4 eq.) Na₂S₂O₄. The reaction was stirred at 55° C., and monitored with TLC (Water:Acetonitrile; 1:2). (R₁=Bn).

### EXAMPLE 10-11

Cleavage of the benzyl group and allyl group, scheme 4.

### EXAMPLE 10

R₁═H (hydrogenolysis of the benzyl group)

XGONHBn (50 g, 36.6 mmol) was dissolved in H₂O (150 mL) and acetic acid was added (3 mL) to obtain a pH of 5. Palladium on activated carbon 10% (2.5 g) was added and the mixture was hydrogenolysed at a pressure of 6-10 psi. The reaction was monitored with MALDI-TOF and after 72 hours the reaction was completed. The catalyst was removed by centrifugation and the clear solution was then concentrated. The products were precipitated in cold ethanol (3 L) and the white material was collected by centrifugation. The precipitate was dissolved in H₂O and freeze dried to give 38 g of the products (R₁═H) (29.7 mmol, 81%).

### EXAMPLE 11

R₁═H (removal of the allyl group)

XGONHAII (0.1 g, 0.076 mmol) was dissolved in a mixture of 3 mL MeOH, 2 mL H₂O and 40 μL of acetic acid. A spatula tip of palladium on activated carbon 10% was added and the mixture was stirred at 60° C. for 48 hours. After completion according to MALDI-TOF the palladium on activated carbon was removed by centrifugation and the clear solution was concentrated and freeze dried to give 85 mg of the products (R₁═H) (0.066 mmol, 87%).

### EXAMPLE 12-13

Xyloglucan with a molecular weight of 15 kD was used, scheme 2.

### EXAMPLE 12

R₁=4,4′-diaminostilbene-2,2′-disulfonic acid (OBA)

Xyloglucan 15 kD (1 g, 0.067 mmol) was dissolved in 20 mL of water (Milli-Q). 4,4′-diaminostilbene-2,2′-disulfonic acid (OBA) (123 mg, 0.33 mmol) and NaCNBH₃ (21 mg 0.33 mmol) were added. The pH was adjusted to pH 5 with 1 M NaOH. The mixture was stirred at 55° C. The course of the reaction was monitored by GPC and after 48 h all the starting material was consumed. The mixture was centrifuged and the clear solution was concentrated and made basic (pH 9) with NH₃ (25%). The product was precipitated in ethanol (500 mL) and the precipitate was collected by centrifugation (4400 rpm). The precipitation was repeated to remove all the 4,4′-diaminostilbene-2,2′-disulfonic acid. The product was then dissolved in water and the ethanol was evaporated before freeze drying to give 990 mg (97%) of the product (R₁=4,4′-diaminostilbene-2,2′-disulfonic acid).

### EXAMPLE 13

R₁=benzyl group (Bn)

Xyloglucan 15 kD (1 g, 0.067 mmol) was dissolved in 15 mL of water. Benzylamine (36 μL, 0.33 mmol) and NaCNBH₃ (21 mg 0.33 mmol) were added. Acetic acid was added to obtain a pH of 5 in the reaction mixture. The mixture was stirred at 55° C. for 18 h and the product was precipitated in ethanol and collected by centrifugation and the precipitation was repeated. The product was dissolved in water and the ethanol was evaporated before freeze drying to give 900 mg (89%) of the product (R₁=Bn).

### EXAMPLE 14-16

Xyloglucan with a molecular weight of 4 KDa (XGO₃) was used, scheme 2.

### EXAMPLE 14

R₁=4,4′-diaminostilbene-2,2′-disulfonic acid (OBA)

XGO₃ (1 g, 0.25 mmol) was dissolved in 20 mL of MeOH/water (2:1). 4,4′-diaminostilbene-2,2′-disulfonic acid (OBA) (463 mg, 1.25 mmol) and NaCNBH₃ (79 mg 1.25 mmol) were added. The pH was adjusted to pH 5 with 1 M NaOH. The mixture was stirred at 55° C. The course of the reaction was monitored by GPC and after 18 h all the starting material was consumed. The mixture was centrifuged and the clear solution was concentrated to remove the methanol. The solution was made basic (pH 9) by the addition of NH₃ (25%). The product was precipitated in ethanol (500 mL) and the precipitate was collected by filtration. The filtrate was washed extensively with ethanol before it was dissolved in water and concentrated. The solution was freeze dried to give 1.0 g (98%) of the product (R₁=4,4′-diaminostilbene-2,2′-disulfonic acid)

### EXAMPLE 15

R₁=benzyl group (Bn)

XGO₃ (1 g, 0.25 mmol) was dissolved in 20 mL of MeOH/water (2:1). Benzylamine (136 μL, 1.25 mmol) and NaCNBH₃ (79 mg 1.25 mmol) were added. Acetic acid was added to obtain a pH of 5 in the reaction mixture. The mixture was stirred at 55° C. and after 18 h the reaction was completed according to MALDI-TOF. The mixture was concentrated and the products were precipitated in ethanol (500 mL). The mixture was filtrated and the precipitate was washed with ethanol and then dissolved in water. Concentration followed by freeze drying gave 970 mg (96%) of the product (R₁=Bn).

### EXAMPLE 16

R₁=dodecyl

XGO₃ (1 g, 0.25 mmol) was dissolved in 20 mL of MeOH/water (1:1). Dodecylamine (140 mg, 0.75 mmol) and NaCNBH₃ (50 mg 0.75 mmol) were added. Acetic acid was added to obtain a pH of 5 in the reaction mixture. The mixture was stirred at 55° C. for 18 h. More dodecylamine (140 mg) and NaCNBH₃ (50 mg) were added and the reaction was stirred at 55° C. for 18 h. The reaction was completed according to MALDI-TOF. The mixture was concentrated and the product was precipitated in acetone. The white material was collected by centrifugation, dissolved in water, concentrated and freeze dried to give 0.71 g of the product (71%). (R₁=dodecyl).

TABLE 1
Summary of reductive amination reactions on carbohydrates presented in the literature

| | Referens | | | | |
|---|---|---|---|---|---|
| | | Brumer et al | Ponpipom et al. | | Evangelista et al. |
| | WO2004/094646 | 2004 | 1980 | WO98/15566 | 1996 |
| Carbohydrate | XGO | XGO | D- | Digested | glucose |
| used | | | mannose | dextrane | and NAc- |
| | | | | or | glucose |
| | | | | xyloglucan | |
| No: of | 7-54 | 7-9 | 1 | 1 or 7-9 | 1 |
| monosaccharide | | | | | |
| units | | | | | |
| Branched | Yes | yes | no | yes | no |
| carbohydrates | | | | | |
| Mw (average) | 1350-6500 | 1350 | 340 | 180- | 180 |
| carbohydrate | | | | 1350 | |
| Method and | Dis. in | Dis. in | Dis. in | NaCNBH₃ | Dis. in |
| reagents used | acetate | NH₄HCO₃. | EtOH | Additional | water |
| in the 1^{st} step | buf. | (saturate | with | reagents | ATPS** |
| | Aniline, | d) | BnNH₂ | and | NaCNBH₃ |
| | NaCNBH₃, | NaCNBH₃ | reflux | conditions | Acetic |
| | Inc. at 70° | Inc. at | for 5 | unclear | acid Inc. |
| | C., 4 h. | RT, 7 | min. | | at 75° C., |
| | | days | | | 1 h. |
| pH in reaction | pH 4.5 | Not | Not | pH 6 or | Pref. |
| | | presented | presented | greater | below pH |
| | | | | | 3 |
| Pre-purifica- | Cool to | None | None | None | Cool to |
| tion step | R.T. | | | | R.T. |
| Purification | Dialysis | Filtration | Crystal- | Chromatog- | |
| before step 2 | | | lisation | raphy | |
| Method and | Incubate | Add | | | |
| reagents used | with | acetic | | | |
| in the 2^{nd} step | diazonium | acid to | | | |
| | salt | pH2 | | | |
| | solution at | | | | |
| | 4° C. 18 h | | | | |
| Final | Chromatog- | Chromatog- | | | |
| purification | raphy | raphy | | | |
| Yield: | <50%? | 51% | N.P. | N.P. | N.P |

| | Referens | | | | |
|---|---|---|---|---|---|
| | | Larm and | | | Danielsson |
| | Yoshida and | Scholander | Kagan | | and Gray |
| | Lee 1994 | 1977 | 1957 | EP0725082 | 1986 |
| Carbohydrate | Lactose | Dextrane, | galactose | Solid corn | cellobiose, |
| used | Laminari- | agarose | and | syrup | cellotetraose, |
| | hexalose | Sepharose | glucose | | cellopentaose |
| | Manno- | | | | |
| | pentalose | | | | |
| No: of | 2 | variable | 1 | — | 2 |
| monosaccharide | | | | | |
| units | | | | | |
| Branched | No | yes | no | yes | no |
| carbohydrates | | | | | |
| Mw (average) | 340 | 10-150k | 180 | 750 to | 340 |
| carbohydrate | | | | 3300 | |
| Method and | Dissolved in | Dissolved | Dissolved | Dissolved | Dissolved |
| reagents used | 25% MeOH, | in water | in water | in water, | in |
| in the 1^{st} step | add a mixture | NaCNBH₃ | with | 40% | K₃PO₄ |
| | of BnNH₂ and | Acetic | BnNH₂ at | methylamine, | buffer, |
| | glacial acetic | acid Inc | 60° C., | Ni- | Inc. at |
| | acid, | at RT, 3 | add | catalyst, | 37° C. for |
| | Inc. at 55° C., | days | MeOH | H₂ at 700 | 6 to 20 |
| | 14 h | | and PtO, | psi, 50° C., | days |
| | | | add H₂ at | 12 h | |
| | | | 60° C., 15 | | |
| | | | h | | |
| pH in reaction | pH 7* | pH 6.5 | Not | Not | pH 8.0 |
| | | | discussed | discussed | |
| Pre-purifica- | Cool to R.T | N.P. | Cool to | N.P. | Dialysis |
| tion step | | | R.T. | | |
| Purification | Extraction | N.P | Crystal- | Filtration | |
| before step 2 | and | | lisation | and | |
| | chromatog- | | | concentrated | |
| | raphy | | | | |
| Method and | Palladium on | N.P | Hydrogenated | Dodecyl | |
| reagents used | activated | | with | isocyanate, | |
| in the 2^{nd} step | carbon 10% | | Palladium | HCl, ion | |
| | | | on | exchange | |
| | | | activated | | |
| | | | carbon | | |
| | | | 10% | | |
| Final | | N.P | Crystal- | Freeze | |
| purification | | | lisation | dry | |
| Yield: | 23 to 95%*** | 1.9 to 6.9% | 42 to 49% | N.P. | 75% |

*The pH optimum is pH 7, pH 6-9 tested in this article. The pH optimum should be 8-9 according to Schwartz & Gray 1977 BnNH₂ = Benzylamine
**ATPS = 8-aminopyrene-1,3,6-trisulfonate
***Depending on reagent and reaction conditions.
R.T. = room temperature N.P. Not Presented

1. A method to produce an aminated water soluble carbohydrate in a reaction mixture comprising a water soluble carbohydrate and a primary amine under reducing conditions
I) where the reaction mixture is made acidic (adjusted to a pH<7) and then incubated,
II) then the generated aminated water soluble carbohydrate is precipitated.

2. The method according to claim 1, wherein the primary amine has the formula R₁NH₂, wherein R₁ is a chemical group removable or not removable from the amine.

3. The method according to claim 1, further comprising the steps of removing the R₁ group and creating a primary amino group on the carbohydrate.

4. The method according to claim 1, further comprising the steps of removing the R₁ group and creating a primary amino group on the carbohydrate by
III) hydrogenolysing the generated aminated carbohydrate when R₁ is a benzyl group by
a) dissolving the generated aminated carbohydrate in water and acid and reacting it with hydrogen gas at an elevated pressure in the presence of an catalyst,
b) removing the catalyst
c) where after the aminated water soluble carbohydrate product may be precipitated.

5. The method according to claim 1, further comprising the
steps of removing the R₁ group and creating a primary amino group on the carbohydrate by
IV) isomerisation and hydrolysis of the generated aminated carbohydrate when the R₁ group is an allyl group.
a) dissolving the generated aminated carbohydrate in water and acid and reacting it in the presence of an catalyst,
b) removing the catalyst
c) where after the aminated water soluble carbohydrate product may be precipitated.

6. The method according to claim 1, wherein the reducing condition in step I) is a hydrogen atmosphere and a catalyst selected from the group consisting of platinum, platinum derivatives and a reducing agent that reduces imines and enamines.

7. The method according to claim 1 wherein the amine is an primary organic amine selected from the group consisting of benzylamine, allylamine, dodecylamine, 4-nitroaniline and 4,4′-diaminostilbene-2,2′-disulfonic acid.

8. The method according to claim 1, wherein the water soluble carbohydrate is hemicellulose.

9. The method according to claim 8, wherein the water soluble carbohydrate is xyloglucan oligosaccharide.

10. The method according to claim 1, wherein an alcohol is added in the reaction mixture before reaction in step I).

11. The method according to claim 1, wherein the pH in the reaction mixture in I) is adjusted to about 5.

12. The method according to claim 1, wherein step II) is modified such that after the optional addition of an alcohol and substantial completion of the reaction, the alcohol is removed.

13. A method to produce an aminated water soluble carbohydrate in a reaction mixture comprising a water soluble carbohydrate and an amine with the formula R₁NH₂, wherein R is a chemical group removable from an amine,
I) where the reaction mixture is incubated,
II) then the generated aminated water soluble carbohydrate may be precipitated,
removing the R₁ group e.g. a benzyl group from the amine by
III) hydrogenolysing the generated aminated carbohydrate by
a) dissolving the generated aminated carbohydrate in water and acid and reacting it with hydrogen gas at an elevated pressure in the presence of an catalyst,
b) removing the catalyst
c) where after the aminated water soluble carbohydrate product may be precipitated or removing the R₁ group from the amine by
IV) isomerisation of the aminated carbohydrate to an enamine and removing the enamine by acid hydrolysis.

14. An aminated hemicellulose molecule, with a molecular weight of above 6400 Da.

15. The aminated hemicellulose molecule according to claim 14, which is an OBA-xyloglucan molecule with a molecular weight of at least 1 kDa.

16. An aminated hemicellulose molecule having the formula: 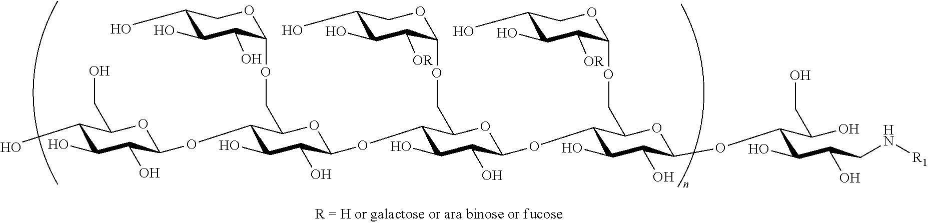
wherein:
R₁ is any chemical group and where R is H, galactose, arabinose or fucose.
n is above 1 such as between 2 and 2000 and the molecular weight is above 1350 Da
n is between 2 and 2000
and the molecular weight is above 6500 Da.

17. The method according to claim 16, wherein the reducing agent that reduces imines and enamines is selected from the group consisting of sodium cyanoborhydride, sodium dithionite and amine borane complexes.

18. The method according to claim 17, wherein the reducing agent that reduces imines and enamines is an amine borane complex selected from the group consisting of pyridine borane, dimethyl amine borane, and 2-picoline borane.
